(19) United States
(12) Plant Patent Application Publication
(10) Pub. No.: US 2002/0059670 P1
(43) Pub. Date: May 16, 2002

(54) LILY PLANT NAMED 'BERNINI'

(76) Inventor: CEES A. VAN DER VOORT, KATWIJI (NL)

Correspondence Address: FOLEY & LARDNER, Washington Harbour, 3000 K Street, N.W., Suite 500, WASHINGTON, DC, 20007-5109, US

(21) Appl. No.: 09/357,103

(22) Filed: Jul. 19, 1999

(51) Int. Cl. A01H 5/00

(52) U.S. Cl. PLT/315

(57) ABSTRACT

A new and distinct cultivar of Lily plant named ‘Bernini’ characterized by having large, upright, pink flowers, curled axis of the outer tepals, dark-pink spots on the tepals, and vigorous growth habit.

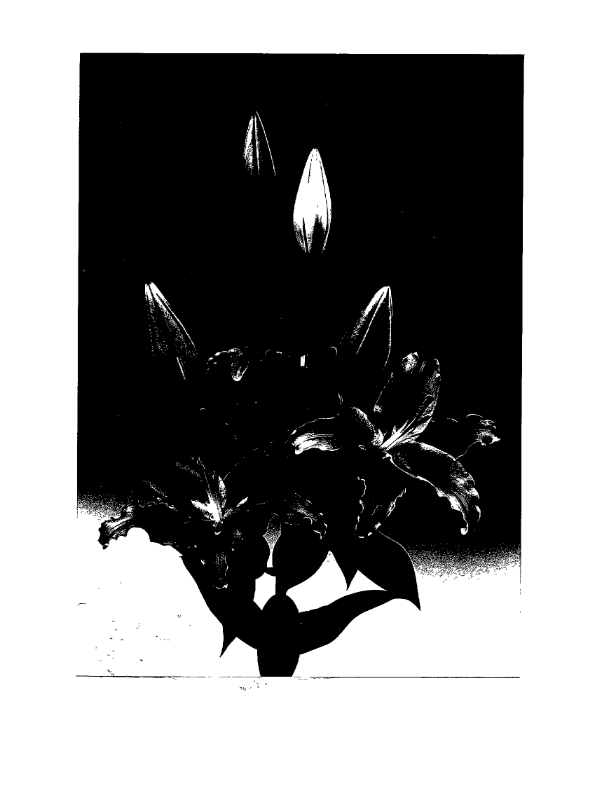

### BACKGROUND OF THE INVENTION

The present invention comprises a new and distinct cultivar of lily plant, botanically known as Lilium L., and hereinafter referred to by the cultivar name ‘Bernini’.

‘Bernini’ is a product of a planned breeding program which had the objective of creating new lily cultivars having large, upright, pink flowers.

‘Bernini’ was originated from a hybridization made by the inventor in a controlled breeding program in Rijnsburg, the Netherlands, in 1983. The male and female parents were unnamed seedlings of Lilium L. oriental hybrids. Both parents are proprietary cultivars used in the breeding program.

‘Bernini’ was discovered and selected as one flowering plant within the progeny of the stated cross by the inventor, Cees A. van der Voort, on Jun. 7, 1983 in a controlled environment in Rijnsburg, the Netherlands.

The first act of asexual reproduction of ‘Bernini’ was accomplished when scales were taken from the initial selection in October 1986 in a controlled environment in Rijnsburg, the Netherlands by, or under the supervision of, Cees A. van der Voort. Horticultural examination of selected units initiated in May, 1986 has demonstrated that the combination of characteristics as herein disclosed for ‘Bernini’ are firmly fixed and are retained through successive generations of asexual reproduction.

### BRIEF DESCRIPTION OF THE INVENTION

The following traits have been repeatedly observed and are determined to be basic characteristics of ‘Bernini’ which, in combination, distinguish this lily as a new and distinct cultivar:

1. large, upright, pink flowers;

2. curled axis of the outer tepals;

3 . dark-pink spots on the tepals; and

4. vigorous growth habit.

‘Bernini’ has not been observed under all possible environmental conditions. The phenotype of the new cultivar may vary significantly with variations in environment such as temperature, light intensity, and daylength without any change in genotype. The following observations, measurements, and comparisons describe plants grown in the Dutch experimental research station CPRO-DLO, Wageningen, the Netherlands under conditions which approximate those generally used in commercial practice.

Of the many commercial cultivars known to the present inventor, no other cultivars are similar in comparison to ‘Bernini’.

### BRIEF DESCRIPTION OF THE DRAWING

The accompanying color photographic drawing shows typical plant, bulb and flower characteristics of ‘Bernini’, with colors being as true as possible with illustrations of this type.

### DETAILED BOTANICAL DESCRIPTION

In the following description, color references are made to The Royal Horticultural Society Colour Chart (RHS). The color values were determined between 12 am and 2 pm at the Dutch experimental research station CPRO-DLO, Wageningen, the Netherlands.

Origin: Seedling from breeding program

Parentage:

Female parent.—Unnamed seedling

Male parent.—Unnamed seedling

Classification:

Botanical.—A hybrid of the genus Lilium L.

i Commercial.—Oriental Lily cv. ‘Bernini’

Plant:

Form.—A single stem carrying numerous, alternately arranged pedicels

Height.—Medium to high for the mature plant, approximately 120 cm

Growth habit.—Vigorous

Stem.—Circular in transverse section; anthocyanin pigmentation absent; internodal length uniform

Foliage.—Quantity: Approximately 70, depending on cultivation conditions Size of leaf: Mature leaves are approximately 11 cm long and 3.5 cm wide Shape of leaf: Generally ovate Color: Light green, RHS 136B

Bulb.—Size: 16 to 18 cm in circumference Color: White/pink

Bud:

Form.—Tapering like a lancet

Size.—12 cm in length depending on cultivation conditions

Opening.—Unfolds normally

Color.—Pink, RHS 64D, with greyish-brown accents

Tepals.—Folded, three visible

Flower:

Blooming habit.—Raceme

Size.—Large, 23 cm in diameter depending on cultivation conditions

Borne.—Upright along a longitudinal axis

Shape.—Generally, hexagonal star in form and bowl-shaped in cross-section

Tepalage.—Number of Tepals: 6 Arrangement: Three inner and three outer Color: Upper Surface: Pink, RHS 63B to RHS 86B Lower Surface: Pink, RHS 65A Spotting or Marking: Spots and markings are present, dark pink in color, RHS 68A

Pedicel.—Length: 11 cm Color: Form:

Texture.—Papillose

Disease resistance.—Medium

Fragrance.—Weak

Lasting quality.—Excellent

Reproductive Organs:

Stamens and anthers.—Arrangements: Anthers form a generally circular pattern at their upper ends Number: 6 Length: Long, depending on cultivation conditions Filaments Color: White

Pollen color.—Greyed-red, RHS 180B

Anther color.—Reddish brown

Pistils.—Green

Stigma.—Dark-pink

Ovaries.—Triangular in shape

I claim:

1. A new and distinct cultivar of Lily plant named ‘Bernini’, as illustrated and described herein.
